(19) United States
(12) Patent Application Publication
(10) Pub. No.: US 2024/0066021 A1
(43) Pub. Date: Feb. 29, 2024

(54) METHODS FOR OPTIMIZING CFTR-MODULATOR THERAPY

(71) Applicant: Children's Hospital Medical Center, Cincinnati, OH (US)

(72) Inventors: Assem G. Ziady, Newport, KY (US); Emily J. Skala, Cleves, OH (US); Maureen B. Dunn, Cincinnati, OH (US); Karen Lammers, North Bend, OH (US)

(21) Appl. No.: 18/272,176

(22) PCT Filed: Jan. 14, 2022
(86) PCT No.: PCT/US22/12561

§ 371 Date: Jul. 13, 2023

Related U.S. Application Data
(60) Provisional application No. 63/212,321, filed on Jun. 18, 2021.
(60) Provisional application No. 63/183,817, filed on May 4, 2021.
(60) Provisional application No. 63/138,030, filed on Jan. 15, 2021.

(51) Int. Cl. A61K 31/47; A61K 31/4025; A61K 31/44; A61K 31/4439; A61K 45/06; A61P 29/00; C12Q 1/6883

(52) U.S. Cl. CPC A61K 31/47; A61K 31/4025; A61K 31/44; A61K 31/4439; A61K 45/06; A61P 29/00; C12Q 1/6883; C12Q 2600/106

(57) ABSTRACT

Disclosed are methods for the treatment of cystic fibrosis in an individual in need thereof. The methods may include the administration of a CFTR modulator, with or without and one or more CFTR modulator therapy optimizing agent. Further disclosed are methods for treating cystic fibrosis in an individual in need thereof which employ detection of one or more biomarkers which may be used to distinguish CFTR modulator responders and non-responders, which may in turn be used to direct therapy in an individual having cystic fibrosis.

### CROSS-REFERENCE TO RELATED APPLICATIONS

This application claims the benefit of, and priority to, U.S. Provisional Application No. 63/138,030, filed Jan. 15, 2021, U.S. Provisional Application No. 63/183,817, filed May 4, 2021, and U.S. Provisional Application No. 63/212,321, filed Jun. 18, 2021, the contents of each are incorporated by reference in their entirety for all purposes.

### STATEMENT REGARDING FEDERALLY-SPONSORED RESEARCH

This invention was made with government support under HL 142210 awarded by the National Institutes of Health. The government has certain rights in the invention.

### BACKGROUND

CF is a recessive heritable disease that affects ˜30,000 individuals in the United States (70,000 globally). CF is a multi-organ disorder with CFTR protein expression in a wide range of tissues throughout the body. There are over 2,000 disease-causing mutations which fall into 5 mutation classifications: biosynthetic defects, misprocessed/mislocalized, defective channel gating or conductance, and improper mRNA splicing. Most patients (˜90%) have at least one F508del (misprocessing) allele (3). The predominant source of morbidity and mortality continues to be lung disease progression. Loss of CFTR function induces pathological changes in chloride and bicarbonate transport and enhanced sodium absorption at the airway surface leading to chronic infection and pathological structural remodeling, including mucus obstruction, airway wall thickening and eventually permanent airway dilation/bronchiectasis. Structural remodeling encourages prolonged and repeated infection and inflammation, feeding a vicious cycle that results in progressive lung function deterioration. Preservation of lung function is useful for reducing morbidity and mortality (4). Over the last decade, there has been significant advancement in CF care, specifically focused on CFTR modulation, with some modulators being highly effective therapies. Despite improvements in treatment of CF patients, further development is needed, in particular for those patients who are not responsive to currently available therapies.

### BRIEF SUMMARY

Disclosed are methods for the treatment of cystic fibrosis in an individual in need thereof. The methods may include the administration of a CFTR modulator, with or without and one or more CFTR modulator therapy optimizing agent. Further disclosed are methods for treating cystic fibrosis in an individual in need thereof which employ detection of one or more biomarkers which may be used to distinguish CFTR modulator responders and non-responders, which may in turn be used to direct therapy in an individual having cystic fibrosis.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Pathway analysis of differential plasma protein expression prior to ivacaftor initiation comparing lung function responders and unsustained responders (standard filter with 5 PSM cutoff). GeneGo™ pathway analysis software was used to analyze protein expression differences in cystic fibrosis subjects that exhibited a response >5% in FEV₁ six months after drug initiation using a standard 5 PSM cutoff. These analyses revealed alterations in responses to wound healing and structural remodeling.

FIG. 2. Pathway analysis of differential plasma protein expression at baseline comparing lung function responders and non-responders. GeneGo™ pathway analysis software was used to analyze protein expression differences in cystic fibrosis subjects that exhibited a response >5% in FEV₁ six months after drug initiation using 20 PSM stringent cutoff. These analyses revealed alterations in ciliary movement, inflammation, and remodeling.

FIG. 3. Pathway analysis of differential plasma protein expression one month following ivacaftor initiation comparing lung function responders and non-responders. GeneGo™ pathway analysis software was used to analyze protein expression differences in cystic fibrosis subjects that exhibited a response >5% in FEV₁ six months after drug initiation using 5 PSM standard cutoff. These analyses revealed alterations in responses to stress and catecholamines.

FIG. 4. Pathway analysis of differential plasma protein expression one month following ivacaftor initiation comparing lung function responders and non-responders. GeneGo™ pathway analysis software was used to analyze protein expression differences in cystic fibrosis subjects that exhibited a response >5% in FEV₁ six mo. after drug initiation using a stringent filter 20 PSM. This analysis highlighted differences in response to drug, inflammation, and cell proliferation and migration.

FIG. 5. Pathway analysis of differential plasma protein expression at baseline comparing lung function responders and non-sustained responders. GeneGo™ pathway analysis software was used to analyze protein expression differences in cystic fibrosis subjects that exhibited a response >5% in FEV₁ six months after drug initiation using a standard 5 PSM standard cutoff. These analyses revealed alterations in WNT signaling, cell movement, and ion transport.

FIG. 6. Pathway analysis of differential plasma protein expression at 6 months comparing lung function responders and non-responders. GeneGo™ pathway analysis software was used to analyze protein expression differences in cystic fibrosis subjects 6 months after CFTR modulation therapy that exhibited a response >5% in FEV₁ six months after drug initiation using a standard 20 PSM stringent cutoff. These analyses revealed alterations in inflammation, positive regulation of nitrogen compound metabolic process, and negative regulation of nitrogen compound metabolic process.

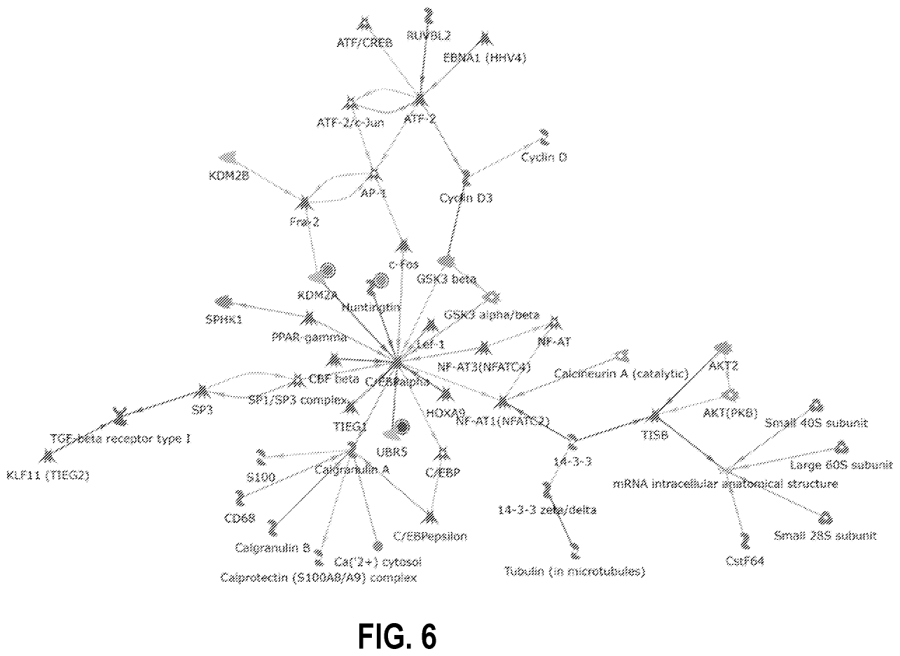

FIG. 7. Pathway analysis of differential plasma protein expression at baseline comparing lung function responders and non-sustained responders. GeneGo™ pathway analysis software was used to analyze protein expression differences in cystic fibrosis subjects that exhibited a response >5% in FEV₁ six months after drug initiation using a standard 20 PSM stringent cutoff. These analyses revealed alterations in inflammation, and positive regulation of nitrogen compound metabolic process.

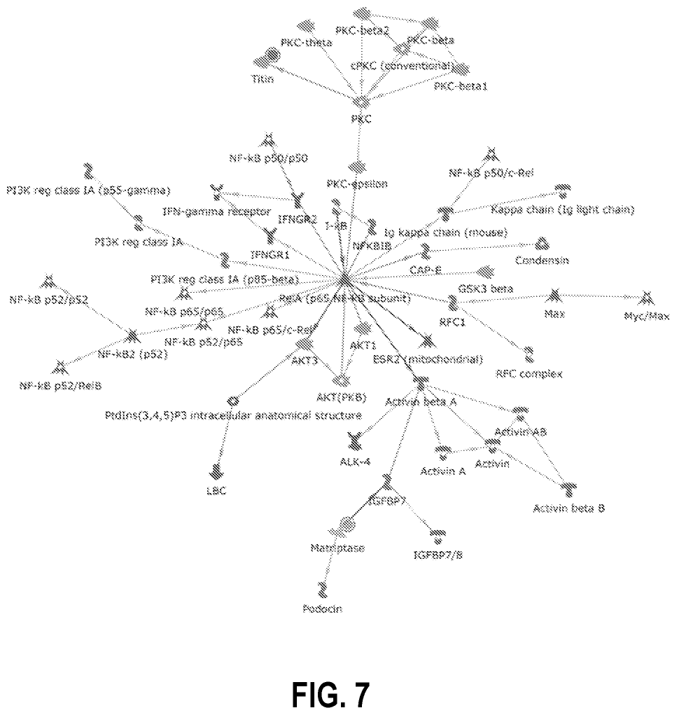

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

With the introduction of triple combination therapy, 90% of individuals with cystic fibrosis (CF) may benefit from highly effective cystic fibrosis transmembrane conductance regulator (CFTR) modulators. In phase three clinical trials, both in F508del heterozygotes and homozygotes, percent predicted Force Expiratory Volume in 1 Second (ppFEV₁) improved by 10-13.8% (depending on study) four weeks after initiation of elexacaftor/tezacaftor/ivacaftor (1, 2). However, approximately 20% of subjects in the triple combination therapy groups in these studies did not have improvement in pulmonary function with ppFEV₁ change ≤5.

Disclosed herein are methods which may be used to improve treatment of CF patients, in particular, by administration of a CFTR modulator therapy optimizing agent to an individual in need thereof. In one aspect, the CF patient may be a patient that is non-responsive or under-responsive to triple combination therapy. In a further aspect, the disclosed methods may be used to identify a patient likely to benefit from the administration of a CFTR modulator therapy optimizing agent a CFTR modulator therapy optimizing agent, for example, by detecting protein levels, which may be used to predict response to high efficacy CFTR modulators, and that may be used to guide therapy, maximizing benefit from the disclosed CFTR modulator therapy optimizing agents in combination with traditional CFTR modulating compounds. Disclosed are biomarkers that may be used to identify pathological differences between subjects with and without lung function response to modulators. Using liquid chromatography with tandem mass spectrometry (LC-MS/MS) and pathway analysis of blood samples obtained from patients with G551D-mediated CF initiated on ivacaftor, Applicant found that proteins in pathways involved in structural remodeling (p=1.52e-18) and inflammation (p=6.81e-6) may be used to predict response to ivacaftor. Discovery analysis indicates that responders and non-responders can be segregated even before initiation of therapy.

Ivacaftor was the first FDA approved CFTR modulator for use in humans and set the standard for highly effective CFTR modulation. It is a CFTR potentiator that acts to increase the open probability of CFTR present at the membrane, initially approved for individuals with at least one G551D gating mutation. In the GOAL study, a longitudinal observational cohort of patients with at least one G551D mutation starting ivacaftor, six months after drug initiation there was a mean change in ppFEV1 6.7 (95% CI 4.9-8.5), a mean change in weight of 2.5 kg (95% CI 1.9-3.1), a mean decline in sweat chloride −54 mEq (95% CI −57.7 to −49.9), a decreased rate of hospitalization (p<0.001) and an improvement in quality of life scores (5). A significant portion (˜50%) of subjects in the study that exhibited physiologic CFTR correction, as evidenced by sweat chloride shifts by −40 to −80 mEq/L, did not have improvement in FEV1>5% (poor lung function response). Patients who do not have improvement in FEV1 within the first month of drug initiation do not have a statistically significant difference in their rate of decline when compared to those with FEV1 improvement after initiation (6). Therefore, the contribution of initial FEV1 improvement is believed to play a significant role in long-term preservation of lung function. In longer term follow up, ivacaftor continues to provide benefit in decreasing pulmonary exacerbations, hospitalizations, and maintain improved BMI (7). Lung function decline is slowed in patients treated with ivacaftor versus patients untreated with CFTR modulators, but lung function continues to decline with age (7). Some evidence suggests that the rate of lung function decline on therapy worsens with time (See reference (8)).

Recently, FDA approval of the promising elexacaftor/tezacaftor/ivacaftor therapy has expanded use of highly effective CFTR modulator therapy. The triple combination includes two CFTR correctors with different binding sites to improve protein folding and presentation at the cell surface as well as a potentiator (1, 2). With clinical trials including both F508del heterozygotic and homozygotic patients, ˜90% of patients now stand to benefit from this therapy. In phase 3 clinical trials, the average increase in ppFEV₁ was 10 (95% CI 7.4-12.6) in homozygotes and 13.6 (95% CI 12.4-14.8) in heterozygotes 4 weeks after therapy initiation with concurrent improvement in BMI, decrease in pulmonary exacerbation frequency, decreased sweat chloride concentration, and improved quality of life scores (1, 2). In these trials, ˜20% of subjects had a change in ppFEV₁≤5 after triple combination therapy initiation. As with the GOAL study, some stable FEV₁ after drug initiation is due to ceiling effect, a significant proportion of patients with poor responses have mild-severe decrease in baseline lung function.

Along with CFTR modulators, advancements in CF therapy have increased the median predicted survival of CF patients to 48 years, and 56% of CF patients alive today are adults (Cystic Fibrosis Foundation-Patient Registry (CFF-PR)-2019). Now with broader application of highly effective modulators of CFTR, these outcomes are expected to continue to improve. However, given the importance of lung function preservation in CF, addressing poor FEV1 responses to therapy is of high interest. In particular, a significant portion of patients with eligible mutations may not derive maximum benefit from modulator therapies. Thus, understanding biological markers that predict poor lung function responses to modulator therapy may be used to define potential CFTR modulator therapy optimizing agent to broaden the benefit. In one aspect, phenotyping protein expression in blood before and after initiation of therapy may be used to improve treatment of CF patients. Population studies in diseases other than CF indicate that protein expression in blood can reflect intracellular and extracellular processes in the lung and other organs (9-15).

The problem of poor lung function response to high efficacy modulators of CFTR is significant for CF care, preventing some patients from fully benefiting from a major therapy for the disease. The disclosed methods provide for improved therapeutic benefit via administration of pathway analysis-identified CFTR modulator therapy optimizing agent. In further aspects, the disclosed methods provide for stratification of patients into CFTR modulator responders and non-responders, based on detection of one or more biomarkers. Based on responder/non-responder status, the appropriate therapy may be administered to the CFTR patient, allowing for personalized therapy and/or streamlining of treatment, improving both efficacy and, where therapies are minimized, improved compliance.

In one aspect, a method for treating cystic fibrosis in an individual in need thereof is disclosed. The method may comprise administering a CFTR modulator and a CFTR modulator therapy optimizing agent to an individual in need thereof. In one aspect, the individual may be characterized as a “CFTR modulator non-responder.” The CFTR modulator therapy optimizing agent may be co-administered with the CFTR modulator to the individual.

In one aspect, the CFTR modulator therapy optimizing agent may be co-administered with said CFTR modulator, wherein the co-administration is carried out for the duration of said CFTR modulator administration.

In one aspect a method for treating cystic fibrosis in an individual in need thereof, is disclosed, in which the method may comprise detecting a level of one or more biomarkers selected from T-cell factor/lymphoid enhancer-binding factor (Tcf (lef)) proteins, WNT, LRP5, DKK1, Frizzled, Ep-CAM, vimentin, MMP-7, VEGF-A, VEGFR-2, EGFR, claudin-1, STAT1/STAT3 regulated signaling of mTORC1, Stabilin-2, angiopoietin 1, Matriptase, RNF213, and G alpha (q), SOX17 regulated proteins, including SRGAP1, GMIP, Guanylate cyclase, SLMAP, EDEM3, CLN2, FAM130A2, Nsp3, MUNC13, TUBGCP5, a beta-catenin regulated protein, PTPN13 (FAP-1) protein phosphatase, matriptase, Ephrin-B receptor 1, BIRC2 (c-IAP1), LDH, phosphodiesterase E (PDE), PDE7a, NOX, NOX5, PI3K, PI3KCG; and classifying an individual as a CFTR modulator responder (“a responder”) or a CFTR modulator non-responder (“responder”). In one aspect, a CFTR modulator therapy may be administered to a responder. Where an individual is characterized as a responder, the administration may be free of a secondary therapy, for example, free of one or more c-therapies as described herein.

In one aspect, a method for treating cystic fibrosis in an individual in need thereof is disclosed, wherein the method may comprise detecting a level of one or more biomarkers selected from T-cell factor/lymphoid enhancer-binding factor (Tcf (lef)) proteins, WNT, LRP5, DKK1, Frizzled, Ep-CAM, vimentin, MMP-7, VEGF-A, VEGFR-2, EGFR, and claudin-1; classifying an individual having an increase in said level of said one or more biomarkers as a CFTR modulator non-responder; and administering an anti-inflammatory agent to said CFTR modulator non-responder, wherein said anti-inflammatory is administered concomitantly with said CFTR modulator therapy throughout the duration of said CFTR modulator therapy. Some examples of CFTR modulators include Kalydeco® (ivacaftor), lumacaftor/ivacaftor (marketed as Orkambi®), tezacaftor/ivacaftor (marketed as Symdeko®), elexacaftor/tezacaftor/ivacaftor (Trikafta™), VX-659, VX-445, VX-152, VX-440, VX-371, VX-561, VX-659, GLPG1837, GLPG2222, GLPG2737, GLPG2451, GLPG1837, PTI-428, PTT-801, PTT-808, eluforsen, and combinations thereof. The detecting may be carried out prior to a CFTR modulator treatment, during a CFTR modulator treatment, or both.

In one aspect, the anti-inflammatory therapy may be selected from one or more of ibuprofen, a steroid, a statin, or combination thereof. The level may include an increase in post-translational modification of said one or more biomarker. In one aspect, the increase in the aforementioned biomarker is an indicator of organ injury, remodeling, angiogenesis, and cellular adhesion.

In one aspect, a method for treating cystic fibrosis in an individual in need thereof is disclosed, wherein the method may comprise detecting a level of one or more biomarkers selected from STAT1/STAT3 regulated signaling of mTORC1, Stabilin-2, angiopoietin 1, Matriptase, RNF213, and G alpha (q); and administering an anti-inflammatory agent to an individual having an increase in said level of said one or more biomarkers. The detected level may include an increase in post-translational modification of said one or more biomarker. The increase in the level may be an indicator of one or more of lung tubal development, ciliary movement, mucus clearance, and antigen presentation. In one aspect, the anti-inflammatory therapy may be selected from one or more of ibuprofen, steroids, and a statin. The method may further include administering a mucolytic therapy, for example, Dornase alfa, hypertonic saline or combinations thereof.

In one aspect, a method for treating cystic fibrosis in an individual in need thereof is disclosed, wherein the method may comprise detecting a level of one or more biomarkers selected from SOX17 regulated proteins, including SRGAP1, GMIP, Guanylate cyclase, SLMAP, EDEM3, CLN2, FAM130A2, Nsp3, MUNC13, and TUBGCP5; and administering an anti-inflammatory agent to an individual having an increase in said level. Such biomarkers may be an indicators of mucin production and secretion, remodeling, CFTR activation, catecholamine response to stress, vascular smooth muscle tone, and degradation of misfolded proteins. In this aspect, an anti-inflammatory therapy may be selected from a non-steroidal anti-inflammatory, a steroid, a statin, and combinations thereof. The method may include administering a mucolytic therapy, such as, for example, Dornase alfa, hypertonic saline or combinations thereof. The level may include an increase in post-translational modification of said one or more biomarker.

In one aspect, a method for treating cystic fibrosis in an individual in need thereof is disclosed, wherein the method may comprise detecting a level of one or more of a beta-catenin regulated protein; and administering an anti-inflammatory therapy to an individual having an increase in said level of said one or more biomarkers. Said biomarkers may be indicators of ciliary structure, remodeling, and inflammation. In one aspect, the anti-inflammatory therapy may be selected from a nonsteroidal anti-inflammatory, a steroid, a statin, and combinations thereof. The level may include an increase in post-translational modification of said one or more biomarker.

In one aspect, a method for treating cystic fibrosis in an individual in need thereof is disclosed, wherein the method may comprise detecting a level of PTPN13 (FAP-1) protein phosphatase; and administering a therapy that targets PTPN13 to an individual having an increase in said level of said one or more biomarkers. In one aspect, the therapy that targets PTPN13 may be selected from rapamycin, FK506, glutathione, and combinations thereof. The level may include an increase in post-translational modification of PTPN13 (FAP-1) protein phosphatase.

In one aspect, a method for treating cystic fibrosis in an individual in need thereof is disclosed, wherein the method may comprise detecting a level of matriptase as an indicator of nitrogen compound metabolism; and administering a therapy that targets matriptase to an individual having an increase in said level of said one or more biomarkers. The therapy that targets matriptase may be selected from pentamidine, WXUK1, and combinations thereof. The level may include an increase in post-translational modification of matriptase.

In one aspect, a method for treating cystic fibrosis in an individual in need thereof is disclosed, wherein the method may comprise detecting a level of Ephrin-B receptor 1 as an indicator of nitrogen compound metabolism; and administering a therapy that targets Ephrin-B receptor 1 to an individual having an increase in said level of Ephrin-B receptor 1. The therapy that targets Ephrin-B receptor 1 may be selected from Erdafitinib, Fedratinib, Neflamapimod, and combinations thereof. The level may include an increase in post-translational modification of Ephrin-B receptor 1.

In one aspect, a method for treating cystic fibrosis in an individual in need thereof is disclosed, wherein the method may comprise detecting a level of BIRC2 (c-IAP1) as an indicator of nitrogen compound metabolism; and administering a therapy that targets BIRC2 (c-IAP1) to an individual having a decrease in said level of said BIRC2 (c-IAP1). The therapy that targets BIRC2 (c-IAP1) may be selected from AT-406, GDC-0152, and combinations thereof. The level may include an increase in post-translational modification of BIRC2 (c-IAP1).

In one aspect, a method for treating cystic fibrosis in an individual in need thereof is disclosed, wherein the method may comprise detecting LDH as an indicator of nitrogen compound metabolism; and administering a therapy that targets LDH to an individual having decrease in said level of LDH. In one aspect, the therapy that targets LDH may be verapamil. The level may include a change in the level of protein having a post-translational modification.

In one aspect, a method for treating cystic fibrosis in an individual in need thereof is disclosed, wherein the method may comprise detecting one or more of a biomarker selected from phosphodiesterase E (PDE), and PDE7a as an indicator of nitrogen compound metabolism; and administering a therapy that targets PDE to said individual having a change in PDE and/or PDE7a level, as compared to baseline. In one aspect, the therapy that targets PDE may be selected from one or more of a nonselective PDE inhibitors (methylated xanthines and derivatives, caffeine, aminophylline, IBMX (3-isobutyl-1-methylxanthine), paraxanthine, pentoxifylline, theobromine, theophylline, a PDE2 selective inhibitor selected from EHNA (erythro-9-(2-hydroxy-3-nonyl)adenine), BAY 60-7550 (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), Oxindole, PDP (9-(6-Phenyl-2-oxohex-3-yl)-2-(3,4-dimethoxybenzyl)-purin-6-one), a PDE3 selective inhibitor selected from Inamrinone, milrinone, Enoximone Anagrelide, Cilostazol, Pimobendan, a PDE4 selective inhibitor selected from Mesembrenone, Rolipram, Ibudilast, Piclamilast, a PDE5 selective inhibitor selected from Sildenafil, tadalafil, vardenafil, udenafil, Dipyridamole, Luteolin, Drotaverine, Roflumilast, Apremilast, Crisaborole, a quinazoline type PDE7 selective inhibitor, a PDE9 selective inhibitor such as Paraxanthine, a PDE10 selective inhibitor such as Papaverine. The level may include an increase in protein having a post-translational modification.

In one aspect, a method for treating cystic fibrosis in an individual in need thereof is disclosed, wherein the method may comprise detecting one or both of NOX and NOX5, as an indicator of nitrogen compound metabolism; and administering a therapy that targets NOX activity to an individual having an increase in NOX and/or NOX 5 as compared to baseline. The therapy that targets NOX activity may be selected from one or more of GKT136901, GKT137831(Setanaxib), diphenyleneiodonium (DPI), apocynin, ebselen, VAS2870, Diapocynin, GSK2795039, and combinations thereof. The level may include an increase in protein having a post-translational modification.

In one aspect, a method for treating cystic fibrosis in an individual in need thereof is disclosed, wherein the method may comprise detecting PI3K, (for example, PI3KCG), as an indicator of nitrogen compound metabolism; and administering a therapy that targets PI3K activity to an individual having a change in PI3K level as compared to a baseline level. The therapy that targets PI3K activity may be selected from one or more of LY294002, Sonolisib, TG100115, Alpelisib, AMG-319, and combinations thereof. The level may include a change in the level of PI3K protein having a post-translational modification.

The disclosed methods may employ testing of any biological fluid in which the levels of protein are detectable and indicative of a therapeutic response to any of the disclosed therapies. In one aspect, the level employed in the methods is a protein level detected in the blood of an individual. The detection of the proteins, or post-translational modification of a protein, may be carried out by any known method, for example, ELISA, mass spectrometry proteomics, or a combination thereof.

In one aspect, the term “CFTR modulator” may include an agent or compound that modulates (for example, increases) the activity of CFTR; in certain specific aspects, the CFTR modulator increases the activity of a CFTR protein. The increase in activity resulting from a CFTR modulator may include compounds that correct, potentiate, stabilize and/or amplify CFTR. “CFTR modulator” may include CFTR correctors, CFTR potentiators, CFTR stabilizers, and CFTR amplifiers. A CFTR corrector is an agent or compound that increases the amount of functional CFTR protein to the cell surface, resulting in enhanced ion transport. A CFTR potentiator is an agent or compound that increases the channel activity of CFTR protein located at the cell surface, resulting in enhanced ion transport. A CFTR stabilizer results in an elongated presence of CFTR in the epithelial cell membrane. A CFTR amplifier is an agent that enhances the effect of a CFTR potentiator, corrector, and/or stabilizer. That co-therapies described herein may be used to provide a benefit when used in combination with a CFTR modulator.

CFTR modulator therapy optimizing agent. In one aspect, the CFTR modulator therapy optimizing agent may be selected from an anti-inflammatory agent (for example, an NSAID (non-steroidal anti-inflammatory), for example, ibuprofen, a steroid, a statin, and combinations thereof), a mucolytic therapy (for example, Dornase alfa, hypertonic saline or combinations thereof), a beta-catenin regulated protein inhibitor such as cambinol, a therapy that targets PTPN13 (for example, rapamycin, FK506, glutathione, and combinations thereof), a therapy that targets matriptase (for example, pentamidine, WXUK1, and combinations thereof), a therapy that targets Ephrin-B receptor 1 (for example, Erdafitinib, Fedratinib, Neflamapimod, and combinations thereof), a therapy that targets BIRC2 (c-IAP1) (for example, AT-406, GDC-0152, and combinations thereof), verapamil, a PDE inhibitor such as Nonselective PDE inhibitors such as methylated xanthines and derivatives, caffeine, aminophylline, IBMX (3-isobutyl-1-methylxanthine), paraxanthine, pentoxifylline, theobromine, theophylline, a PDE2 selective inhibitor selected from EHNA (erythro-9-(2-hydroxy-3-nonyl)adenine), BAY 60-7550 (2-R3,4-dimethoxyphenyl)methyl]-7-1(1R)-1-hydroxyethyl]-4-phenylbutyl]-5-methyl-imidazo[5,1-f][1,2,4]triazin-4(1H)-one), Oxindole, PDP (9-(6-Phenyl-2-oxohex-3-yl)-2-(3,4-dimethoxybenzyl)-purin-6-one), a PDE3 selective inhibitor selected from Inamrinone, milrinone, Enoximone Anagrelide, Cilostazol, Pimobendan, a PDE4 selective inhibitor selected from Mesembrenone, Rolipram, Ibudilast, Piclamilast, a PDE5 selective inhibitor selected from Sildenafil, tadalafil, vardenafil, udenafil, Dipyridamole, Luteolin, Drotaverine, Roflumilast, Apremilast, Crisaborole, a quinazoline type PDE7 selective inhibitor, a PDE9 selective inhibitor such as Paraxanthine, a PDE10 selective inhibitor such as Papaverine) an agent that targets NOX activity (such as one or more of GKT136901, GKT137831(Setanaxib), diphenyleneiodonium (DPI), apocynin, ebselen, VAS2870, Diapocynin, GSK2795039, and combinations thereof), a therapy that targets PI3K activity (for example, one or more of LY294002, Sonolisib, TG100115, Alpelisib, AMG-319, and combinations thereof). In one aspect, one or more CFTR modulator therapy optimizing agent as listed above may be administered prior to a CFTR modulator therapy, at the initiation of CFTR modulator therapy, one month after initiation of a CFTR modulator therapy, three months after initiation of a CFTR modulator therapy, or six months after initiation of a CFTR modulator therapy.

In a further aspect, the CFTR modulator therapy optimizing agent may be one or more selected from Table 1, Table 2, or Table 3, as provided herein. In one aspect, one or more CFTR modulator therapy optimizing agent of Table 1 may be administered prior to administration of a CFTR modulator therapy, at the initiation of CFTR modulator therapy, one month after initiation of a CFTR modulator therapy, three months after initiation of a CFTR modulator therapy, or six months after initiation of a CFTR modulator therapy. In one aspect, one or more CFTR modulator therapy optimizing agent of Table 2 may be administered prior to a CFTR modulator therapy, at the initiation of CFTR modulator therapy, one month after initiation of a CFTR modulator therapy, three months after initiation of a CFTR modulator therapy, or six months after initiation of a CFTR modulator therapy. In one aspect, one or more CFTR modulator therapy optimizing agent of Table 3 may be administered prior to a CFTR modulator therapy, at the initiation of CFTR modulator therapy, one month after initiation of a CFTR modulator therapy, three months after initiation of a CFTR modulator therapy, or six months after initiation of a CFTR modulator therapy.

TABLE 1

| | Gene | | | |
|---|---|---|---|---|
| Drug | Symbol | Target | Effect | Pubmed |
| AZD8055 | MTOR | mTOR | Inhibition | 20028854 |
| Everolimus | MTOR | mTOR | Inhibition | 16061672, |
| | | | | 16217558, |
| | | | | 16443261, |
| | | | | 16731750, |
| | | | | 16390278, |
| | | | | 16652094, |
| | | | | 16908864 |
| Evofosfamide | TXNRD1 | TXNRD1 | Inhibition | 19838642 |
| LY294002 | PIK3CG | PI3Kcatclass | Inhibition | 11294389, |
| | | IB(p110-gamma) | | 15658870, |
| | | | | 15664519, |
| | | | | 16789742, |
| | | | | 17049248 |
| Motexafin | TXNRD1 | TXNRD1 | Inhibition | 16481328 |
| gadolinium | | | | |
| Ridaforolimus | MTOR | mTOR | Inhibition | 12864941, |
| | | | | 14770419, |
| | | | | 15365568, |
| | | | | 16205124, |
| | | | | 16217558 |
| Sirolimus | MTOR | mTOR | Inhibition | 14508096, |
| | | | | 17041628, |
| | | | | 12217904 |
| Sonolisib | PIK3CG | PI3Kcatclass | Inhibition | 16170026 |
| | | IB(p110-gamma) | | |
| Temsirolimus | MTOR | mTOR | Inhibition | 16790088, |
| intracellular | | | | 18413763 |
| TG100115 | PIK3CG | PI3Kcatclass | Inhibition | 17172449 |
| | | IB(p110-gamma) | | |

TABLE 2

| | Gene | | | |
|---|---|---|---|---|
| Drug | Symbol | Target | Effect | Pubmed |
| Ketamine | GRIN2A | NR2A | Inhibition | 8336337, |
| | | | | 8941398, |
| | | | | 9719604, |
| | | | | 11438305, |
| | | | | 11937336 |
| Memantine | GRIN2A | NR2A | Inhibition | 16563064, |
| | | | | 16809810, |
| | | | | 16809811, |
| | | | | 16854944, |
| | | | | 16912819, |
| | | | | 17132970, |
| | | | | 17157509, |
| | | | | 17624774, |
| | | | | 16009352, |
| | | | | 17112636, |
| | | | | 17123715 |
| Remacemide | GRIN2A | NR2A | Inhibition | 9023271 |
| BI-2536 | PLK1 | PLK1 | Inhibition | 17291758, |
| | | | | 18005335 |
| Indantadol n | GRIN2A | NR2A | Inhibition | 11378157 |
| Isosorbide | NPR1 | Guanylatecyclase | Inhibition | |
| dinitrate | | A(NPR1) | | |
| LY294002 | PIK3CG | PI3K catclass | Inhibition | 11294389, |
| | | IB(p110-gamma) | | 15658870, |
| | | | | 15664519, |
| | | | | 16789742, |
| | | | | 17049248 |
| Nesiritide | NPR1 | Guanylatecyclase | Activation | |
| | | A(NPR1) | | |
| Sonolisib | PIK3CG | PI3K catclass | Inhibition | 16170026 |
| | | IB(p110-gamma) | | |
| TG100115 | PIK3CG | PI3K catclass | Inhibition | 17172449 |
| | | IB(p110-gamma) | | |
| Tofacitinib | JAK3 | JAK3 | Inhibition | 21383241, |
| | | | | 14593182, |
| | | | | 15491777, |
| | | | | 18183025, |
| | | | | 19361440, |
| | | | | 20138049, |
| | | | | 20478313, |
| | | | | 21105711, |
| | | | | 21144599, |
| | | | | 22037378 |

TABLE 3

| | Gene | | | |
|---|---|---|---|---|
| Drug | Symbol | Target | Effect | Pubmed |
| Ketamine | GRIN2A | NR2A/NR3A | Inhibition | 8336337, |
| | | | | 8941398, |
| | | | | 9719604, |
| | | | | 11438305, |
| | | | | 11937336 |
| Memantine | GRIN2A | NR2A/NR3A | Inhibition | 17084865, |
| | | | | 8336337, |
| | | | | 8941398, |
| | | | | 9719604, |
| | | | | 11937336 |
| Almotriptan | HTR1B | HTR1B | Activation | 11134654, |
| | | | | 15762767, |
| | | | | 15853532, |
| | | | | 16236092, |
| | | | | 16625988, |
| | | | | 16971345, |
| | | | | 16995333 |
| Bardoxolone | IKBKB | IKK-beta | Inhibition | |
| methyl | | | | |
| Foretinib | AXL | UFO | Inhibition | 19671800 |
| Frovatriptan | HTR1B | HTR1B | Activation | 9986723 |
| GSK-923295 | CENPE | CENP-E | Inhibition | |
| Indantadol | GRIN2A | NR2A | Inhibition | 11378157 |
| MLN0415 | IKBKB | IKK-beta | Inhibition | |
| Naratriptan | HTR1B | HTR1B | Activation | 9801818, |
| | | | | 9986723 |
| Prednisolone | SERPINA6 | SERPINA6 | Unspecified | |
| Remacemide | GRIN2A | NR2A | Inhibition | 9023271 |
| Rizatriptan | HTR1B | HTR1B | Activation | 9229132, |
| | | | | 9357514, |
| | | | | 9986723, |
| | | | | 10417495, |
| | | | | 11472239, |
| | | | | 12814962 |
| Sipatrigine | SCN2A | SCN2A | Inhibition | 12130650 |
| Tetrodotoxin | SCN2A | SCN2A | Inhibition | 1325650, |
| | | | | 3754035 |
| Xanthohumol | IKBKB | IKK-beta | Inhibition | 18952893 |

Dosage

The methods may include orally administering to a cystic fibrosis patient one or more of the aforementioned CFTR modulator therapy optimizing agentsat a therapeutically effective dose, for example, a dose at which the CFTR modulator therapy is enhanced. In one aspect, the cystic fibrosis patient is being treated with a CFTR modulator therapy.

In one aspect, the CFTR modulator therapy optimizing agent listed in Table 1, or Table 2, or Table 3, is administered to the cystic fibrosis patient at a total daily dose of from about 200 mg to about 1 mg, or from about 100 mg to about 5 mg, or from about 50 mg to about 10 mg, or less, or from about 25 mg to about 20 mg. In one aspect, the cystic fibrosis patient may be undergoing concomitant CFTR modulator therapy (regardless of lung disease phenotype) wherein a therapeutically effective amount of a CFTR potentiator and/or a CFTR corrector is concomitantly administered to said patient. In one aspect, the CFTR potentiator may be ivacaftor (KALYDECO®). In further aspects, the CFTR correctors may be one or both of lumacaftor and tezacaftor. In further aspects, one CFTR potentiator and at least one CFTR corrector may be administered. For example, a combination including ivacaftor can be administered; a combination of ivacaftor and lumacaftor, for example ORKAMBI® (lumacaftor/ivacaftor) may be administered. In certain aspects, at least two CFTR correctors, or at least one CFTR corrector and at least one CFTR potentiator may be administered. For example, a combination of ivacaftor and lumacaftor, for example, ORKAMBI® (lumacaftor/ivacaftor) may be administered. In other aspects, two CFTR correctors may be administered, optionally with a CFTR potentiator; the combination may, for example, include ivacaftor. The methods further include the use of the disclosed CFTR modulator therapy optimizing agent with triple combination regimens comprising ivacaftor, such as ivacaftor, tezacaftor, and another corrector for the treatment of cystic fibrosis. The method may include administering one or more CFTR modulator therapy optimizing agents with a triple combination regimen, for example, such a triple combination can include tezacaftor plus ivacaftor and one of the following: VX-445, VX-659, VX-440, VX-371, VX-152, GLPG1837, GLPG2222, GLPG2737, GLPG2451, GLPG1837, PTI-428, PTT-801, PTT-808, eluforsen. In other embodiments the triple combination can be comprised of other CFTR modulators. In yet other embodiments the combination may be comprised of four or more such CFTR modulators. As described above, the one or more CFTR modulator therapy optimizing agents may, for example, be administered at a dose of about 10 mg every 12 or 24 hours, or about 20 mg every 12 or 24 hours, or about 25 every 12 or 24 hours, or about 30 mg every 12 or 24 hours, or about 40 mg every 12 or every 24 hours, or about 50 mg every 12 or 24 hours, or at a dose of about 100 mg every 12 or 24 hours.

In one aspect, the CFTR patient is not eligible for treatment with one or more of ivacaftor, lumacaftor, tezacaftor, VX-659, VX-445, VX-152, VX-440, VX-371, VX-561, VX-659 or combinations thereof. In one aspect, the CFTR patient is not eligible for treatment with one or more of ivacaftor, lumacaftor, tezacaftor, VX-659, VX-445, VX-152, VX-440, VX-371, VX-561, VX-659, GLPG1837, GLPG2222, GLPG2737, GLPG2451, GLPG1837, PTI-428, PTT-801, PTT-808, eluforsen, or combinations thereof.

The CFTR modulator therapy optimizing agent may be initiated at the time of the first dose of one or more CFTR modulator therapy initiation, or within one month of the first dose of one or more CFTR modulator therapy initiation, or within three months of the first dose of one or more CFTR modulator therapy initiation, or within six months of the first dose of one or more CFTR modulator therapy initiation.

In one aspect, the methods may be used to treat cystic fibrosis, pulmonary inflammation, chronic lung inflammation, and/or to decrease pulmonary exacerbations in a cystic fibrosis patient in need thereof, comprising administering to said patient one or more CFTR modulator therapy optimizing agent as provided herein. Such patients may include, patients of a mild lung disease phenotype, a moderate lung disease phenotype, or a sever lung disease phenotype.

In one aspect, the patient in need of treatment may be a male or female of 2 years or older, or of 3 years or older, or of 6 years or older, or of 7 years or older, or of 12 years or older, or of 13 years or older, or of 18 years or older, or of 19 years or older, or of 25 years or older, or of 25 years or older, or of 30 years or older, or of 35 years or older, or of 40 years or older, or of 45 years or older, or of 50 years or older. In some embodiments, a patient in need of treatment is less than 50 years old, or less than 45 years old, or less than 40 years old, or less than 35 years old, or less than 30 years old, or less than 25 years old, or less than 20 years old, or less than 19 years old, or less than 18 years old, or less than 13 years old, or less than 12 years old, or less than 7 years old, or less than 6 years old, or less than 3 years old, or less than 2 years old. In one aspect, a patient in need of treatment may be a male or female from 2 to 18 years old, or from 2 to 12 years old, or from 2 to 6 years old, or from 6 to 12 years old, or from 6 to 18 years old, or from 12 to 16 years old, or from 2 to 50 years old, or from 6 to 50 years old, or from 12 to 50 years old, or from 18 to 50 years old. In one aspect, a patient in need of treatment may be a female who is pregnant or who may become pregnant. In one aspect, a patient may have an F508del mutation. In one aspect, the patient may have a homozygous F508del mutation. In one aspect, the patient may have a heterozygous F508del mutation. In one aspect, the patient may not have an F508del mutation. In one aspect, the patient may have had at least one pulmonary exacerbation in the year prior to the first administration of the CFTR modulator therapy optimizing agent. In one aspect, the methods may be used to treat cystic fibrosis, reduce pulmonary inflammation, and/or to treat chronic lung inflammation and/or decreasing pulmonary exacerbations in a cystic fibrosis patient in need thereof.

In a further aspect, the method may comprise administering an additional therapeutic agent (secondary therapy), in addition to the CFTR modulator and the CFTR modulator therapy optimizing agent as described herein. The additional therapeutic agent may be, for example, a drug used in the treatment of cystic fibrosis such as a bronchodilator, an antibiotic, a mucolytic, a surfactant, a pancreatic enzyme replacement drug, or a combination thereof. In other aspects, the additional therapeutic agent (secondary therapy) may be a physical treatment such as an airway clearance therapy.

In one aspect, the additional therapeutic agent (secondary therapy) may be a beta-agonist. Exemplary beta-agonists include albuterol, salbutamol, levalbuterol, formoterol, fenoterol, salmeterol, bambuterol, brocaterol, clenbuterol, terbutalin, tulobuterol, epinephrin, isoprenalin, and hexoprenalin. In another aspect, the yet additional therapeutic agent is an anticholinergic agent. Exemplary anticholinergics are tiotropium, oxitropium, ipratropium, and glycopyrrolate. In a further embodiment, the additional therapeutic agent is a mucolytic and/or a surfactant. Exemplary mucolytics and surfactants are hypertonic saline, normal saline, acetylcystein, ambroxol, carbocystein, tyloxapol, dipalmytoylphosphatidylcholin, recombinant surfactant proteins, and DNase. In one embodiment, the yet additional therapeutic agent is an antibiotic agent. Exemplary antibiotics are beta-lactam antibiotics, including amoxycillin, piperacillin, cephalosporines, including cefaclor, cefazedon, cefuroxim, cefoxitin, cefodizim, cefsulodin, cefpodixim, and cefixim, carbapenemes such as imipenem and cilastatin, monbactames, such as, aztrenonam, aminoglycosides, including streptomycin, neomycin, paromomycin, kanamycin, gentamycin, amicacin, tobramycin, and spectinomycine, tetracyclines, such as doxycyclin and minocycline, macrolides including erythromycine, clarithromycine, roxithromycine, azithromycin, josamycine, and spiramycine, gyrase inhibitors or quinolones such as ciprofloxacin, ofloxacine, levofloxacine, pefloxacine, lomefloxacine, fleroxacine, clinafloxacine, sitafloxacine, gemifloxacine, balofloxacine, trovafloxacine, and moxifloxacine, sulfonamides and nitroimidazoles (including metronidazol, tinidazol), chloramphenicol, lincomycine, clindamycine, and fosfomycine, and glycopeptides such as Vancomycine and Teicoplanine. I

In one aspect, the additional therapeutic agent (secondary therapy) may be an anti-inflammatory drug. Exemplary anti-inflammatory drugs include ibuprofen, dornase alfa, BILL 284, ajulemic acid, a PDE4 inhibitor (e.g., roflumilast), romoglycate and nedocromil.

In one aspect, the additional therapeutic agent (secondary therapy) may be azithromycin.

In one aspect, the additional therapeutic agent (secondary therapy) may be a corticosteroid. Exemplary corticosteroids include beclomethasone, betamethasone, budesonide, ciclesonide, flunisolide, fluticasone, icomethasone, mometasone, rofleponide, and triamcinolone. In yet further aspects, the additional therapeutic agent is bradykinin, prostaglandin, leukotriene and platelet activating factor antagonists.

Administration of the compounds or drugs described herein encompasses administration of a pharmaceutically acceptable salt of a CFTR modulator therapy optimizing agent as described herein. The active agent may form salts, which are also within the scope of the preferred embodiments. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps, which may be employed during preparation. Salts of the compounds of the active agent may be formed, for example, by reacting a compound of the active agent with an amount of acid or base, such as an equivalent amount, in a medium such as one in which the salt precipitates or in an aqueous medium followed by lyophilization. When the compounds are in the forms of salts, they may comprise pharmaceutically acceptable salts. Such salts may include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

In one aspect, active agents provided herein may be administered in a dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

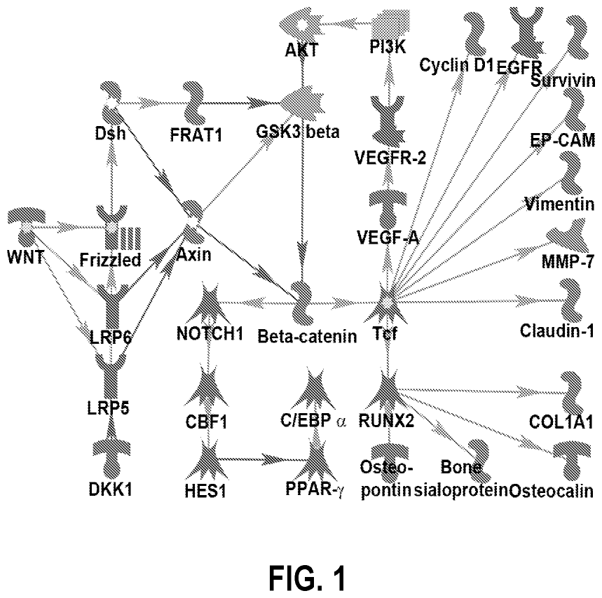

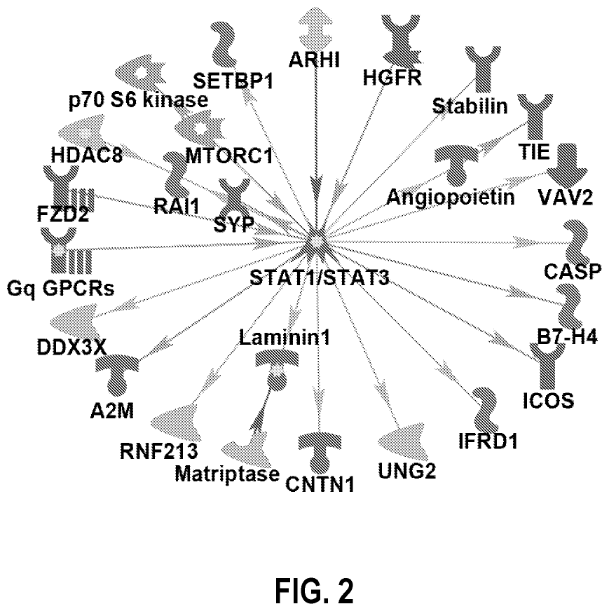

Applicant obtained plasma samples from the GOAL study for subjects with evidence of physiologic response to ivacaftor based on sweat chloride drug response (−60 to −80 mEq) and a baseline ppFEV1 64%-84% to avoid ceiling effect for lung function non-response. Samples from baseline, one, and six months after drug initiation were studied comparing subjects with sustained FEV1 improvement (ΔppFEV1>5) at one and six months (baseline n=15, one month n=15, six months n=14), subjects with poor FEV1 response (ΔppFEV1<5) at both one and six months (baseline n=27, one month n=15, 6 months n=15), and subjects without sustained FEV1 improvement (ΔppFEV1>5 at one month but <5 at six months) (baseline n=4, one month n=3, six months n=2). There were no samples from subjects with FEV1<5 at 1 month but >5 at 6 months in our cohort. Samples were randomized and blinded by the office of clinical and translational research (OCTR) at Cincinnati Children's Hospital Medical Center (CCHMC). OCTR conducted the blinding independent of any members of the study team. Once samples were blinded and randomized, they were depleted of albumin to ensure that less abundant protein signatures could be captured by mass spectrometry (MS) and fractionated using gel and column chromatography. Once all the proteomic data was collected for the samples, OCTR provided us with sample grouping by lung function response to modulator at one and six months. Two cutoffs of proteomic data, based on peptide spectrum match (PSM, a sequencing spectral match for a portion of a protein) were used to filter the data. At baseline there were 904 protein differences (standard 5 PSMs cutoff) and 107 proteins differences (stringent 20 PSMs cutoff). Pathway analysis was used to compare patients based on lung function response to modulator. In comparison of baseline samples from lung function responders and non-responders using a standard 5 PSM cutoff (FIG. 1) there was enrichment for wound healing (p=5.08e-33), cellular organization (p=1.52e-18, FDR=1.75e-15), and migration (p=3.34e-17, FDR=2.30e-14). Using a stringent 20 PSM cutoff, there was differential association with cell cycle regulation (p=8.09e-12), lung tubal development (p=8.86e-10) associated with ciliary movement (p=2.10e-9), antigen processing/immune responses (p=3.68e-6, FDR=6.43e-5), and organelle/cell migration (2.55e-5, FDR=1.03e-2) (FIG. 2). These data suggest that differences in structural remodeling, ciliary dysfunction, and inflammatory signaling segregate patients who will have a lung function response from those that will not respond prior to initiation of therapy.

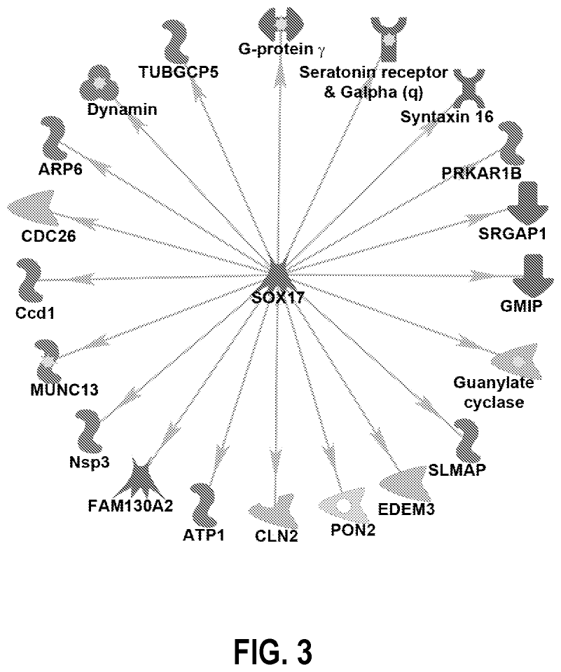

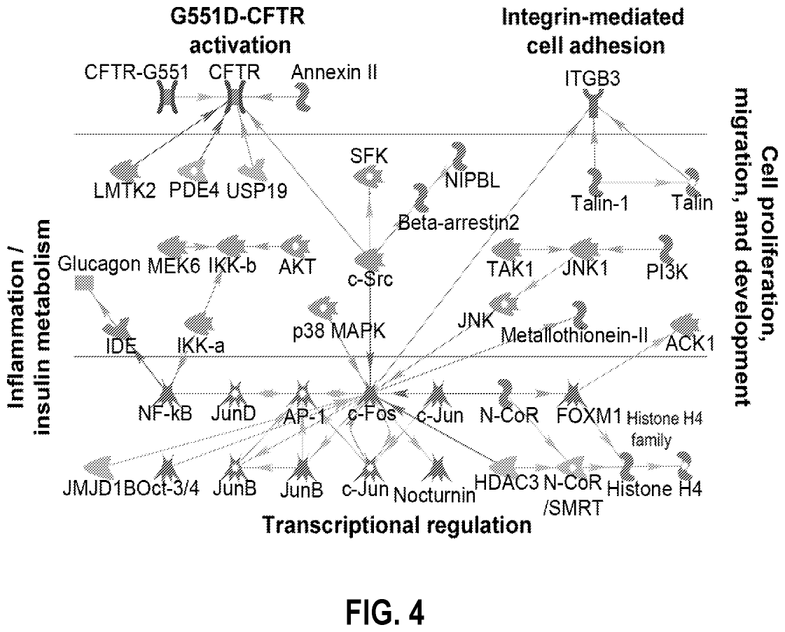

One month after ivacaftor initiation, there were 429 protein differences with a 5 PSM cutoff and 27 protein differences with a 20 PSM cutoff. In the standard 5 PSM cutoff there were significant differences in response to regulation of vascular smooth muscle contraction (p=2.41e-17) and catecholamines (p=6.39e-11) (FIG. 3). With the more stringent 20 PSM cutoff, a G551D-CFTR modulation signature is detectable (p=2.14e-19) (FIG. 4) and differential IL-18-mediated inflammation (p=1.30e-3, FDR=9.5 le-2). These post-ivacaftor initiation analyses indicate that differences between lung function responders and non-responders include changes in G551D CFTR modulation, stress response, and fibrosis, in addition to baseline differences. Six months after ivacaftor initiation, there were 680 protein differences using a standard 5 PSM cutoff and with the stringent 20 PSM cutoff there were 58 differences between groups.

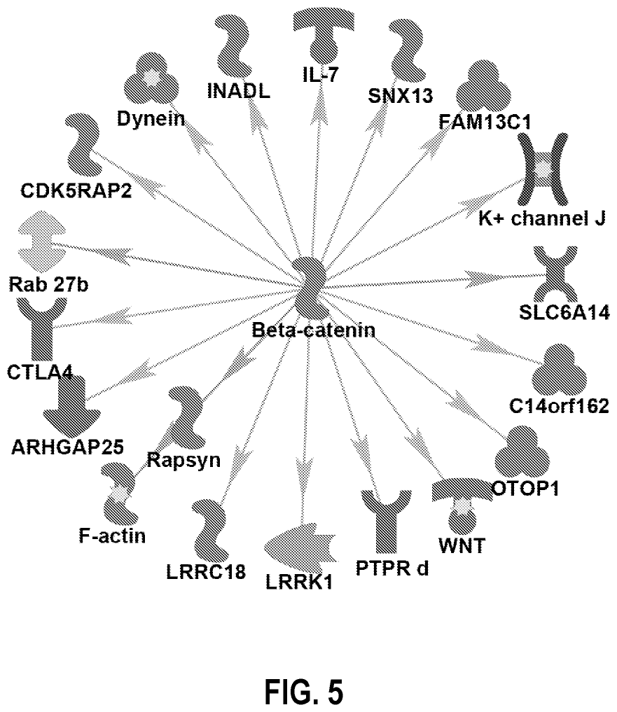

There were a small number of samples from subjects with non-sustained FEV₁ improvement, with change in ppFEV₁>5 at one month, but return to baseline FEV₁ by six months. At baseline, with 5 PSM cutoff we identified 656 protein differences, and with the 20 PSM cutoff there were 95 differences. At baseline, responders and non-sustained responders exhibited differential cellular adhesion (p=1.40e-11, FDR=5.38e-8), lipoprotein metabolism (p=2.40e-6, FDR=5.97e-4), and WNT signaling (p=9.65e-4, FDR=3.99e-2). At one month the standard 5 PSM cutoff had 673 protein differences, with 20 PSM cutoff there were 75 differences. Non-sustained responders exhibited changes in cell adhesion/proliferation (p=4.81e-5, FDR=5.00e-3). Six months post-drug initiation, 5 PSM cutoff identified 315 protein differences, 6 protein differences with the 20 PSM cutoff. Pathway analysis identified differences in non-sustained responses shifted to membrane protein stability (p=2.82e-23), cellular proliferation/motility (p=5.42e-3, FDR=1.05e-2), and ion transport regulation (p=8.69e-3, FDR=1.05e-2) (FIG. 5). The power was limited in these analyses due to a small number of subjects in the group with non-sustained lung function response (baseline n=4, one month n=3, six months n=2). Thus, the data suggest that differences exist between subjects with sustained lung function responses and those that do not, prominently, changes in cholesterol metabolism, wound healing, and cell migration.

### REFERENCES

1. Heijerman H G M, McKone E F, Downey D G, Van Braeckel E, Rowe S M, Tullis E, Mall M A, Welter J J, Ramsey B W, McKee C M, Marigowda G, Moskowitz S M, Waltz D, Sosnay P R, Simard C, Ahluwalia N, Xuan F, Zhang Y, Taylor-Cousar J L, McCoy K S. Efficacy and safety of the elexacaftor plus tezacaftor plus ivacaftor combination regimen in people with cystic fibrosis homozygous for the F508del mutation: a double-blind, randomised, phase 3 trial. Lancet (London, England) 2019; 394: 1940-1948.
2. Middleton P G, Mall M A, Dřevínek P, Lands L C, McKone E F, Polineni D, Ramsey B W, Taylor-Cousar J L, Tullis E, Vermeulen F, Marigowda G, McKee C M, Moskowitz S M, Nair N, Savage J, Simard C, Tian S, Waltz D, Xuan F, Rowe S M, Jain R. Elexacaftor-Tezacaftor-Ivacaftor for Cystic Fibrosis with a Single Phe508del Allele. The New England journal of medicine 2019; 381: 1809-1819.
3. Drumm M L, Ziady A G, Davis P B. Genetic variation and clinical heterogeneity in cystic fibrosis. Annu Rev Pathol 2012; 7: 267-282.
4. Schluchter M D, Konstan M W, Davis P B. Jointly modelling the relationship between survival and pulmonary function in cystic fibrosis patients. Stat Med 2002; 21: 1271-1287.
5. Rowe S M, Heltshe S L, Gonska T, Donaldson S H, Borowitz D, Gelfond D, Sagel S D, Khan U, Mayer-Hamblett N, Van Dalfsen J M, Joseloff E, Ramsey B W. Clinical mechanism of the cystic fibrosis transmembrane conductance regulator potentiator ivacaftor in G551D-mediated cystic fibrosis. Am J Respir Crit Care Med 2014; 190: 175-184.
6. Heltshe S L, Rowe S M, Skalland M, Baines A, Jain M. Ivacaftor-treated Patients with Cystic Fibrosis Derive Long-Term Benefit Despite No Short-Term Clinical Improvement. American journal of respiratory and critical care medicine 2018; 197: 1483-1486.
7. Volkova N, Moy K, Evans J, Campbell D, Tian S, Simard C, Higgins M, Konstan M W, Sawicki G S, Elbert A, Charman S C, Marshall B C, Bilton D. Disease progression in patients with cystic fibrosis treated with ivacaftor: Data from national US and UK registries. Journal of cystic fibrosis: official journal of the European Cystic Fibrosis Society 2020; 19: 68-79.
8. Volkova N, Moy K, Evans J, Campbell D, Tian S, Simard C, Higgins M, Konstan M W, Sawicki G S, Elbert A, Charman S C, Marshall B C, Bilton D. Disease progression in patients with cystic fibrosis treated with ivacaftor: Data from national US and UK registries. J Cyst Fibros 2019.
9. Soares H D, Potter W Z, Pickering E, Kuhn M, Immermann F W, Shera D M, Ferm M, Dean R A, Simon A J, Swenson F, Siuciak J A, Kaplow J, Thambisetty M, Zagouras P, Koroshetz W J, Wan H I, Trojanowski J Q, Shaw L M, Biomarkers Consortium Alzheimer's Disease Plasma Proteomics P. Plasma biomarkers associated with the apolipoprotein E genotype and Alzheimer disease. Arch Neurol 2012; 69: 1310-1317.
10. Geyer P E, Wewer Albrechtsen N J, Tyanova S, Grassi N, Iepsen E W, Lundgren J, Madsbad S, Hoist J J, Torekov S S, Mann M. Proteomics reveals the effects of sustained weight loss on the human plasma proteome. Mol Syst Biol 2016; 12: 901.
11. Wewer Albrechtsen N J, Geyer P E, Doll S, Treit P V, Bojsen-Moller K N, Martinussen C, Jorgensen N B, Torekov S S, Meier F, Niu L, Santos A, Keilhauer E C, Hoist J J, Madsbad S, Mann M. Plasma Proteome Profiling Reveals Dynamics of Inflammatory and Lipid Homeostasis Markers after Roux-En-Y Gastric Bypass Surgery. Cell Syst 2018; 7: 601-612 e603.
12. Krisp C, Parker R, Pascovici D, Hayward N K, Wilmott J S, Thompson J F, Mann G J, Long G V, Scolyer R A, Molloy M P. Proteomic phenotyping of metastatic melanoma reveals putative signatures of MEK inhibitor response and prognosis. Br J Cancer 2018; 119: 713-723.
13. Geyer P E, Kulak N A, Pichler G, Holdt L M, Teupser D, Mann M. Plasma Proteome Profiling to Assess Human Health and Disease. Cell Syst 2016; 2: 185-195.
14. Huttenhain R, Choi M, Martin de la Fuente L, Oehl K, Chang C V, Zimmermann A K, Malander S, Olsson H, Surinova S, Clough T, Heinzelmann-Schwarz V, Wild P J, Dinulescu D M, Nimeus E, Vitek O, Aebersold R. A Targeted Mass Spectrometry Strategy for Developing Proteomic Biomarkers: A Case Study of Epithelial Ovarian Cancer. Mol Cell Proteomics 2019; 18: 1836-1850.
15. Grassi N, Kulak N A, Pichler G, Geyer P E, Jung J, Schubert S, Sinitcyn P, Cox J, Mann M. Ultra-deep and quantitative saliva proteome reveals dynamics of the oral microbiome. Genome Med 2016; 8: 44.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for treating cystic fibrosis in an individual in need thereof, comprising administering a CFTR modulator and a CFTR modulator therapy optimizing agent to said individual.

2. The method of claim 1, wherein said individual is a CFTR modulator non-responder.

3. The method of claim 1 or claim 2, wherein said CFTR modulator therapy optimizing agent is co-administered with said CFTR modulator.

4. The method of any preceding claim, wherein said CFTR modulator therapy optimizing agent is co-administered with said CFTR modulator, wherein said co-administration is carried out for the duration of said CFTR modulator administration.

5. A method for treating cystic fibrosis in an individual in need thereof, comprising
detecting a level of one or more biomarkers selected from T-cell factor/lymphoid enhancer-binding factor (Tcf (lef)) proteins, WNT, LRP5, DKK1, Frizzled, Ep-CAM, vimentin, MMP-7, VEGF-A, VEGFR-2, EGFR, claudin-1, STAT1/STAT3 regulated signaling of mTORC1, Stabilin-2, angiopoietin 1, Matriptase, RNF213, and G alpha (q), SOX17 regulated proteins, including SRGAP1, GMIP, Guanylate cyclase, SLMAP, EDEM3, CLN2, FAM130A2, Nsp3, MUNC13, TUBGCP5, a beta-catenin regulated protein, PTPN13 (FAP-1) protein phosphatase, matriptase, Ephrin-B receptor 1, BIRC2 (c-IAP1), LDH, phosphodiesterase E (PDE), PDE7a, NOX, NOX5, PI3K, PI3KCG;
classifying an individual as a CFTR modulator responder (“a responder”) or a CFTR modulator non-responder (“non-responder”);
wherein a CFTR modulator therapy is administered to a responder; and
wherein said administration is free of one or more secondary therapies.

6. A method for treating cystic fibrosis in an individual in need thereof, comprising detecting a level of one or more biomarkers selected from T-cell factor/lymphoid enhancer-binding factor (Tcf (lef)) proteins, WNT, LRP5, DKK1, Frizzled, Ep-CAM, vimentin, MMP-7, VEGF-A, VEGFR-2, EGFR, and claudin-1;
classifying an individual having an increase in said level of said one or more biomarkers as a CFTR modulator non-responder; and
administering an anti-inflammatory agent to said CFTR modulator non-responder, wherein said anti-inflammatory is administered concomitantly with said CFTR modulator therapy throughout the duration of said CFTR modulator therapy.

7. The method of claim any preceding claim, wherein said CFTR modulator is selected from Kalydeco® (ivacaftor), Orkambi® (lumacaftor/ivacaftor), Symdeko® (tezacaftor/ivacaftor), Trikafta® (elexacaftor/tezacaftor/ivacaftor), and combinations thereof.

8. The method of any preceding claim, wherein said detecting is carried out prior to a CFTR modulator treatment

9. The method of any preceding claim, wherein said detecting is carried out during a CFTR modulator treatment

10. The method of any of claims 6 through 9, wherein said anti-inflammatory agent is selected from one or more of ibuprofen, a steroid, a statin, or combination thereof.

11. The method of any preceding claim, wherein said level includes an increase in post-translational modification of said one or more biomarker.

12. A method for treating cystic fibrosis in an individual in need thereof, comprising detecting a level of one or more biomarkers selected from STAT1/STAT3 regulated signaling of mTORC1, Stabilin-2, angiopoietin 1, Matriptase, RNF213, and G alpha (q); and
administering an anti-inflammatory agent to an individual having an increase in said level of said one or more biomarkers.

13. The method of claim 12, wherein said increase in said level is an indicator of one or more of lung tubal development, ciliary movement, mucus clearance, and antigen presentation.

14. The method of claim 12 or 13, wherein said anti-inflammatory therapy is selected from one or more of ibuprofen, steroids, and a statin.

15. The method of any of claims 12 through 14, further comprising administering a mucolytic therapy.

16. The method of claim 15, wherein said mucolytic therapy is selected from Dornase alfa, hypertonic saline or combinations thereof.

17. The method of any of claims 12 through 16, wherein said level includes an increase in post-translational modification of said one or more biomarker.

18. A method for treating cystic fibrosis in an individual in need thereof, comprising detecting a level of one or more biomarkers selected from SOX17 regulated proteins, including SRGAP1, GMIP, Guanylate cyclase, SLMAP, EDEM3, CLN2, FAM130A2, Nsp3, MUNC13, and TUBGCP5; and
administering an anti-inflammatory agent to an individual having an increase in said level.

19. The method of claim 18, wherein said anti-inflammatory therapy is selected from a non-steroidal anti-inflammatory, a steroid, a statin, and combinations thereof.

20. The method of any of claim 18 or 19, further comprising administering a mucolytic therapy.

21. The method of claim 20 wherein said mucolytic therapy is selected from Dornase alfa, hypertonic saline or combinations thereof.

22. The method of any of claims 19 through 21, wherein said level includes an increase in post-translational modification of said one or more biomarker.

23. A method for treating cystic fibrosis in an individual in need thereof, comprising
detecting a level of one or more of a beta-catenin regulated protein; and
administering an anti-inflammatory therapy to an individual having an increase in said level of said one or more biomarkers.

24. The method of claim 23, wherein said anti-inflammatory therapy is selected from a nonsteroidal anti-inflammatory, a steroid, a statin, and combinations thereof.

25. The method of any of claim 23 or 24, wherein said level includes an increase in post-translational modification of said one or more biomarker.

26. A method for treating cystic fibrosis in an individual in need thereof, comprising
detecting a level of PTPN13 (FAP-1) protein phosphatase; and
administering a therapy that targets PTPN13 to an individual having an increase in said level of said one or more biomarkers.

27. The method of claim 26, wherein said therapy that targets PTPN13 is selected from rapamycin, FK506, glutathione, and combinations thereof.

28. The method of claim 26 or 27 wherein said level includes an increase in post-translational modification of PTPN13 (FAP-1) protein phosphatase.

29. A method for treating cystic fibrosis in an individual in need thereof, comprising
detecting a level of matriptase as an indicator of nitrogen compound metabolism; and
administering a therapy that targets matriptase to an individual having an increase in said level of said one or more biomarkers.

30. The method of claim 29, wherein said therapy that targets matriptase is selected from pentamidine, WXUK1, and combinations thereof.

31. The method of claim 29 or 30 wherein said level includes an increase in post-translational modification of matriptase.

32. A method for treating cystic fibrosis in an individual in need thereof, comprising
detecting a level of Ephrin-B receptor 1 as an indicator of nitrogen compound metabolism; and
administering a therapy that targets Ephrin-B receptor 1 to an individual having an increase in said level of Ephrin-B receptor 1.

33. The method of claim 32, wherein said therapy that targets Ephrin-B receptor 1 is selected from Erdafitinib, Fedratinib, Neflamapimod, and combinations thereof.

34. The method of claim 32 or 33 wherein said level includes an increase in post-translational modification of Ephrin-B receptor 1.

35. A method for treating cystic fibrosis in an individual in need thereof, comprising
detecting a level of BIRC2 (c-IAP1) as an indicator of nitrogen compound metabolism; and
administering a therapy that targets BIRC2 (c-IAP1) to an individual having a decrease in said level of said BIRC2 (c-IAP1).

36. The method of claim 35, wherein said therapy that targets BIRC2 (c-IAP1) is selected from AT-406, GDC-0152, and combinations thereof.

37. The method of claim 35 or 36 wherein said level includes an increase in post-translational modification of BIRC2 (c-IAP1).

38. A method for treating cystic fibrosis in an individual in need thereof, comprising
detecting LDH as an indicator of nitrogen compound metabolism; and
administering a therapy that targets LDH to an individual having decrease in said level of LDH.

39. The method of claim 38, wherein said therapy that targets LDH is verapamil.

40. The method of claim 38 or 39, wherein said level includes a change in the level of protein having a post-translational modification.

41. A method for treating cystic fibrosis in an individual in need thereof, comprising
detecting one or more of a biomarker selected from phosphodiesterase E (PDE), and PDE7a as an indicator of nitrogen compound metabolism; and
administering a therapy that targets PDE to said individual having a change in PDE and/or PDE7a level, as compared to baseline.

42. The method of claim 41, wherein said therapy that targets PDE is selected from one or more of a nonselective PDE inhibitors, a PDE2 selective inhibitor, a PDE3 selective inhibitor, a PDE4 selective inhibitor, a PDE5 selective inhibitor, a quinazoline type PDE7 selective inhibitor, a PDE9 selective inhibitor, a PDE10 selective inhibitor, and combinations thereof.

43. The method of claim 41 or 42, wherein said level includes an increase in protein having a post-translational modification.

44. A method for treating cystic fibrosis in an individual in need thereof, comprising
detecting one or both of NOX and NOX5, as an indicator of nitrogen compound metabolism; and
administering a therapy that targets NOX activity to an individual having an increase in NOX and/or NOX 5 as compared to baseline.

45. The method of claim 44, wherein said therapy that targets NOX activity is selected from one or more of GKT136901, GKT137831(Setanaxib), diphenyleneiodonium (DPI), apocynin, ebselen, VAS2870, Diapocynin, GSK2795039, and combinations thereof.

46. The method of claim 44 or 45, wherein said level includes an increase in protein having a post-translational modification.

47. A method for treating cystic fibrosis in an individual in need thereof, comprising
detecting PI3K as an indicator of nitrogen compound metabolism; and
administering a therapy that targets PI3K activity to an individual having a change in PI3K level as compared to a baseline level.

48. The method of claim 47, wherein said therapy that targets PI3K activity is selected from one or more of LY294002, Sonolisib, TG100115, Alpelisib, AMG-319, and combinations thereof.

49. The method of claim 47 or 48, wherein said level includes a change in the level of PI3K protein having a post-translational modification.

50. The method of any preceding claim, wherein said level is a protein level in blood.

51. The method of any preceding claim, wherein said detecting is carried out by ELISA, mass spectrometry proteomics, or a combination thereof.

52. The method of any preceding claim, wherein said individual is administered one or more CFTR modulator enhancing agents selected from Table 1, Table 2, and Table 3.
